(19) United States
(12) Patent Application Publication
(10) Pub. No.: US 2017/0138969 A1
(43) Pub. Date: May 18, 2017

(54) System And Method For Measuring Permeability Of Drugs/Toxic/Chemical Compounds

(71) Applicant: Indian Institute Of Technology Madras, Chennai (IN)

(72) Inventors: S. Harshal, Chennai (IN); Doble Mukesh, Chennai (IN)

(21) Appl. No.: 15/313,626

(22) PCT Filed: May 22, 2015
(86) PCT No.: PCT/IN2015/000217

§ 371 Date: Nov. 23, 2016

(30) Foreign Application Priority Data
May 23, 2014 (IN) 2550/CHE/2014

(51) Int. Cl. G01N 33/94; G01N 33/92; G01N 15/08

(52) U.S. Cl. CPC G01N 33/94; G01N 15/08; G01N 33/92; G01N 2015/086; G01N 2405/04

(57) ABSTRACT

This invention relates to a system and method for measuring the permeability of drugs/toxin chemical compounds utilizing phospholipid monolayer. A phospholipid monolayer can be deposited onto a cellulose membrane by vertical lifting or Langmuir Schafer technique. The phospholipid monolayer or Langmuir Blodgett (LB) film can be coated on the cellulose membrane or porous support.

### TECHNICAL FIELD

Embodiments are generally related to medicine, pharmaceuticals and chemical industry. Embodiments are also related to measurement of physiochemical properties of biologically active molecules/compounds. Embodiments are particularly related to systems and methods for measuring permeability of drugs, toxins, pesticides, carcinogens, plasma protein binding etc. using lipid layer coated membrane.

### BACKGROUND OF THE INVENTION

Measurement of physicochemical properties, including permeability, in a wide range of high-throughput screening environment plays an important role in the selection of the promising biologically-active molecules for lead optimization in pharmaceutical, toxin chemical and biotechnological research and development. Lipid layers are generally adapted for evaluating the permeability of bio-membranes which mediate various reactions such as, substance and/or energy transfer, metabolism, signal transduction, etc. For example, the permeability of drugs across lipid membranes is closely related to drug delivery mechanisms of oral formulations. It is therefore highly critical to measure the permeability of drugs in developing the pharmaceutical compositions. In addition to the drugs, it is also critical to evaluate the membrane permeability of substances which adversely affect living organisms including toxic substances, pesticides and carcinogenic substances, etc.

Conventionally, the permeability of chemical/pharmaceutical, molecules are measured using techniques such as, Octanol-Water co-efficient and Immobilized Artificial Chromatography based on HPLC. The determination of permeability by such prior art techniques are generally expressed in terms of Log P. Alternative, methods that are well known in the art include solvent based PAMPA assay and cell culture based Caco-2 monolayer assay. Such prior art methods are based on the principal of Boyden chamber assay where the system consists of a donor compartment and acceptor compartment separated by porous membrane.

Langmuir-Blodgett film is typically formed by spreading the fatty acid dissolved in a volatile solvent on water surface free of surface active agents. The resulting lipid monolayer formed at air-water interface is considered a half membrane bilayer, the structure with respect to lipid orientation and molecule packing is well established. The Langmuir-Blodgett films can be transferred onto solid flat or micro-porous substrate by vertical transfer or by Langmuir-Schaefer method. Langmuir-Blodgett films are widely exploited to study the membrane properties and small molecule interaction to obtain mechanistic details of the interaction in applications including water evaporation, gas sensing, sensor head, calculation of area per molecule, characterizing surface activity.

PAMPA assay (Parallel Artificial Membrane Permeability Assay) has been widely used for measuring the in vitro permeability of molecules across artificial membrane barriers containing phospholipid supported by a high-porosity micro filter, separating a solution of test molecules from a solution initially free of them. In the Caco-2 assay approach, the cells are seeded and cultured in the donor well containing polycarbonate/PVDF membrane of 0.45 μm pore size. The culture is further incubated for 21 days in order to form the cell monolayer.

Molecules diffusion into the acceptor well through Caco-2 monolayer at a specified time is determined by standard quantification methods. However, Caco-2 assay is a time consuming and cost consuming approach. Also, the Caco-2 assay approach measures the total permeability including passive and active diffusion. The PAMPA assay approach determines only the passive diffusion. In a typical PAMPA method a defined amount (1-4 %) of lipid or a mixture of lipids is solubilized in hexadecane/octanol and the resulting lipid solution is applied on to the membrane. The presence of organic solvent limits the biomimetic aspect that is essential for an effective permeability determination system.

Based on the foregoing, it is believed that a need exists for an improved system and method for measuring permeability of drugs/toxin chemicals. A need also exists for an improved system and method for measuring permeability of drugs/toxin chemical compounds through lipid layer in order to predict oral ingestion of such compounds into the blood stream, as described in detail herein.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for an improved system and method for determining permeability of drugs/toxin chemical compounds.

It is another aspect of the disclosed embodiments to provide for an improved phospholipid monolayer for determining permeability of drugs/toxin chemical compounds using Boyden Chamber assay free of solvent.

It is further aspect of the disclosed embodiments to provide for an improved method for attaching the membrane containing phospholipid monolayer without damaging the monolayer or the porosity of the membrane.

It is another aspect of the present invention to provide for an improved system and method for measuring permeability of drugs/toxin chemical compounds through lipid layer in order to predict oral ingestion of such compounds into the blood stream.

The aforementioned aspects and other objectives and advantages can now be achieved as described herein. An improved system and method for measuring the permeability of drugs/toxin chemical compounds, is described herein. A phospholipid monolayer (e.g., Di-palmitoyl-phosphotidyl-sn-glycerol choline (DPPC)) can be deposited on to a cellulose membrane (e.g., Cellulose nitrate of 0.22 μm pore size) by vertical lifting technique. Note that the phospholipid monolayer can be a Langmuir Blodgett (LB) which can be coated on the cellulose membrane. Polystyrene donor wells can be fabricated using Polystyrene tubes/Poly acrylic tubes. The permeability of the drug/toxin/chemical compound can be further measured using the phospholipid monolayer by Boyden chamber approach. Such an improved system can maintain its arrangement and is solvent free therefore is adapted for effective measurement of permeability of drugs/toxin chemical compounds through lipid layer in order to predict entry of such compounds into the blood stream.

The phospholipid monolayer can be dissolved in CHCl₃ and spread on water surface contained in a Langmuir-Blodgett film and dried for 10 min in order to permit the CHCl₃ to evaporate and the fatty acid is left behind. The fatty acid molecules cane be compressed at rate of 5 mm/min using Teflon barrier connected to a servomotor. The monolayer can be further compressed to 41 mN/m and allowed to stabilize for 3 min. Finally, the monolayer can be transferred to hydrophilic Cellulose Nitrate membrane 0.22 μm using vertical lifting technique at the rate of 1 mm/minute.

Note that the phospholipid monolayer, 1,2-dipalmitoyl-sn-glycero-3-phosphocholine (DPPC), used herein should not be constituted in any limited sense. Alternatively, monolayer of mixture or extract of lipids, other fatty acids can also be alternatively used in the place of proposed monolayer without limiting the scope of the invention. The monolayer can be mass produced and transferred to any solid substrate. The lipid molecules of the proposed invention are arranged layer by layer by eliminating the solvents by making it similar to cell membrane. Membrane support used can be interchanged with affecting the performance.

The method for attaching the Cellulose Membrane proposed herein do not alter the membrane pore and forms a leak proof seal between the tube and the membrane. Also, the method for attaching the monolayer with the membrane is suitable for a wide range of porous substrates including PVDF, Nylon and Polycarbonate membranes. The membrane can be cut into circles of 0.24 cm² area and one end of the tube can be dipped into chloroform (briefly dipped and excess chloroform was removed). The tubes can be further pressed against the membrane immediately. The use of chloroform alters the surface of polystyrene/poly-acrylic acid making it sticky; as the chloroform evaporates the material solidifies forming a leak-proof seal between the membrane and the tube.

The permeability of the drug/toxin/chemical compound can be effectively measured using the phospholipid monolayer by Boyden chamber approach. A 10 μM aqueous solution of the drug/toxic chemical compound can be taken and a 100 μl of the solution can be placed in the donor well of the present invention. The donor well can be then placed inside an acceptor well containing 200 μl of water. The system can be incubated at 25° C. for 6 hours. The donor well and acceptor wells separated and the concentration of the compound can be determined in order to measure the permeability of the drug/toxic chemical compound. The approach is also capable of predicting the diffusion of drugs/pesticides/carcinogens utilizing less amount of lipid compared to traditional systems. The system and method proposed herein can be therefore a cost effective approach for measuring the permeability of a wide range of drugs and toxic chemical compounds.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a process for deposition of monolayer, in accordance with the disclosed embodiments;

FIG. 2 illustrates a assembled PAMPA plate illustrating the assembled well plate viewed from a acceptor well, in accordance with the disclosed embodiments;

FIG. 3-4 illustrates a graph illustrating the membrane integrity test, in accordance with the disclosed embodiments; and

FIG. 5 illustrates a graph illustrating the equilibration time observed in the present system, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The embodiments now will be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

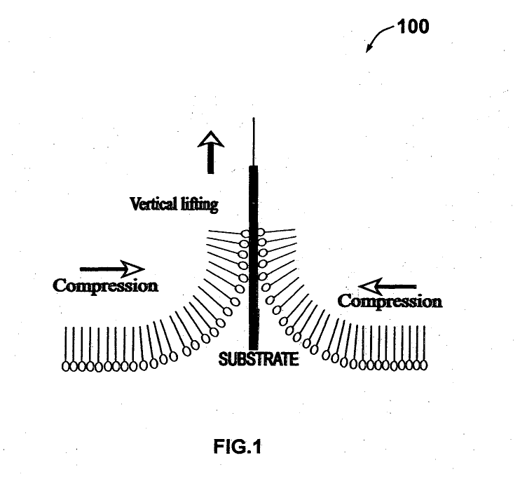

FIG. 1 illustrates a process for deposition of monolayer, in accordance with the disclosed embodiments. The improved system and method proposed herein can be effectively adapted for measurement of permeability of drugs/toxin chemical compounds through lipid layer in order to predict entry of such compounds into the blood stream. The phospholipid monolayer such as, 1,2-dipalmitoyl-sn-glycero-3-phosphocholine (DPPC) can be deposited on to the Nitro cellulose membrane of 0.22 μm pore size by Vertical lifting or Langmuir-schaefer technique. Note that the phospholipid monolayer can be a Langmuir Blodgett (LB) which can be coated on the cellulose membrane. Polystyrene donor wells can be fabricated using Polystyrene tubes/Poly acrylic tubes. Polystyrene donor wells can be fabricated using Polystyrene tubes/Poly acrylic tubes wherein the tubes are pressed against the membrane.

The phospholipid monolayer can be dissolved in CHCl₃ and spread on water surface contained in a Langmuir-Blodgett film and dried for 10 min in order to permit the CHCl₃ to evaporate and the fatty acid is left behind. The fatty acid molecules can be compressed at rate of 5 mm/min using Teflon barrier connected to a servomotor. The monolayer can be further compressed to 41 mN/m and allowed to stabilize for 3 min. Finally, the monolayer can be transferred to hydrophilic Nitrocellulose membrane 0.22 μm using vertical lifting technique at the rate of 1 mm/minute.

Note that the phospholipid monolayer, Di-palmitoyl-phosphotidyl-sn-glycerol choline (DPPC), used herein should not be constituted in any limited sense. Other fatty acids/mixture/lipid extracts can also be alternatively used in the place of proposed monolayer without limiting the scope of the invention. The monolayer can be mass produced and transferred to any solid substrate; stored at room temperature for further use. The lipid molecules of the proposed invention are arranged layer by layer by eliminating the solvents by making it similar to cell membrane.

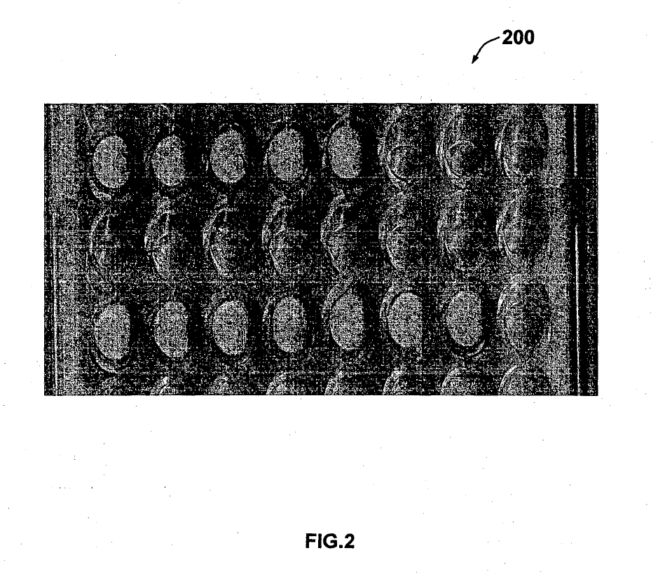

FIG. 2 illustrates an assembled PAMPA plate 200 illustrating the assembled well plate viewed from an acceptor well, in accordance with the disclosed embodiments. The method for attaching the monolayer with the Cellulose Membrane proposed herein do not alter the membrane pore and forms a leak proof seal between the monolayer and the membrane. Also, the method for attaching the monolayer with the membrane is suitable for a wide range of porous substrates including PVDF, Nylon and Polycarbonate membranes. The membrane can be cut into circles of 0.24 cm² area and one end of the tube can be dipped into chloroform (briefly dipped and excess chloroform was removed). The tubes can be further pressed against the membrane immediately. The use of chloroform alters the surface of polystyrene/poly-acrylic acid making it sticky; as the chloroform evaporates the material solidifies forming a leak-proof seal between the membrane and the tube.

The permeability of the drug/toxin/chemical compound can be effectively measured using the phospholipid monolayer by Boyden chamber approach. A 10 μM aqueous solution of the drug/toxic chemical compound can be taken and a 100 μl of the solution can be placed in the donor well of the present invention. The donor well can be then placed inside an acceptor well containing 200 μl of water. The system can be incubated at 25° C. for 6 hours. The donor well and acceptor wells can be separated and the concentration of the compound can be determined in order to measure the permeability of the drug/toxic chemical compound.

The Permeability of the compound can be calculated by flux equation reported by Avdeef et al., (Eur. J. Phar. sci, 14, 271-280; 2001 ):

$$Pe = \frac{- ln [ 1 - \frac{Ca ( t )}{C equilibrium} ]}{A * t * ( 1 / Vd + 1 / Va )}$$

Where; Vd, Va is Donor and acceptor volume
Ca(t) concentration at time ‘t’ in acceptor well
Cd(t) concentration at time ‘t’ in donor well

C_{equilibrium}=[Cd(t)*Vd+Ca(t)*Va]/(Vd+Va)
Area ‘A’, Pe is the permeability (cm/s)

The approach proposed herein can be effectively employed to test plasma-protein binding of drugs by Random Equilibrium Dialysis (RED). The approach is also capable of predicting the diffusion of drugs/pesticides/carcinogens with less amount of lipid compared to traditional systems. The lipid coating has a monolayer of lipid. The lipid coated maintains its arrangement and is solvent free compared to other PAMPA systems. The system and method proposed herein can be therefore a cost effective approach for measuring the permeability of a wide range of drugs and toxic chemical compounds.

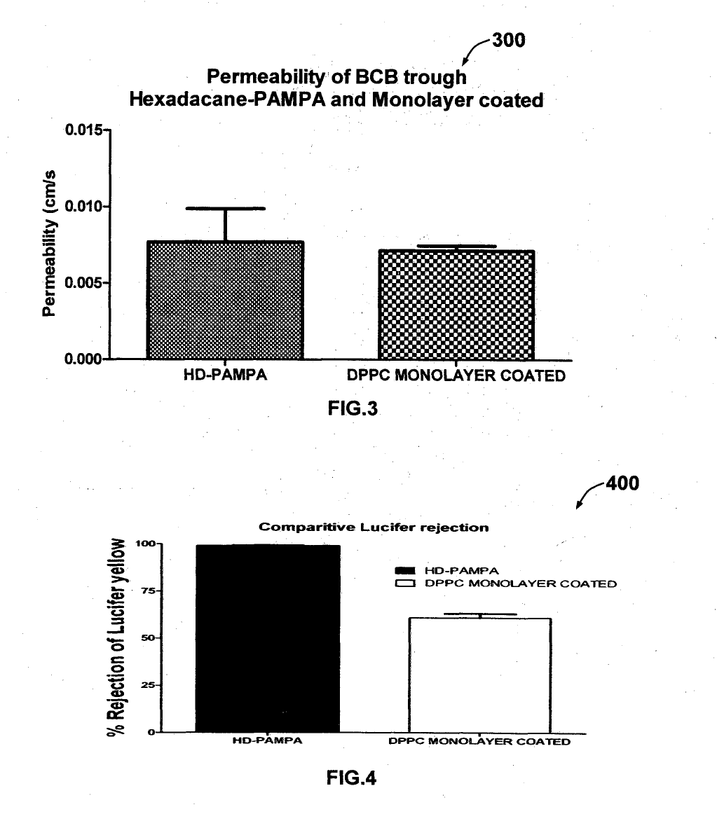

FIG. 3-4 illustrates graphs 300-400 illustrating the membrane integrity test, in accordance with the disclosed embodiments. The membrane integrity test can be performed according to the standard protocol. An 80 μl aqueous solution containing Lucifer yellow (LY) and Brilliant Cresyl Blue (BCB) in the ratio 1:9 is added into the donor well. The PAMPA system can be assembled and incubated for 5 hours at 25° C. The acceptor plate and donor well are separated and concentration of LY and BCB was analyzed by measuring; Fluorescence: Excitation wavelength @428 mn; Emission wavelength @525 nm; Absorbance; at 610 nm for BCB.

FIG. 3 illustrates the graph 300 showing the permeation of BCB as 0.045; Absorbance units after 5 hours of incubation indicating the presence of integral membrane. Similarly, the graph 400 illustrates the rejection of Lucifer Yellow after 5 hours of incubation indicating the presence of integral membrane. The rejection of Lucifer yellow indicates the presence of an integral membrane.

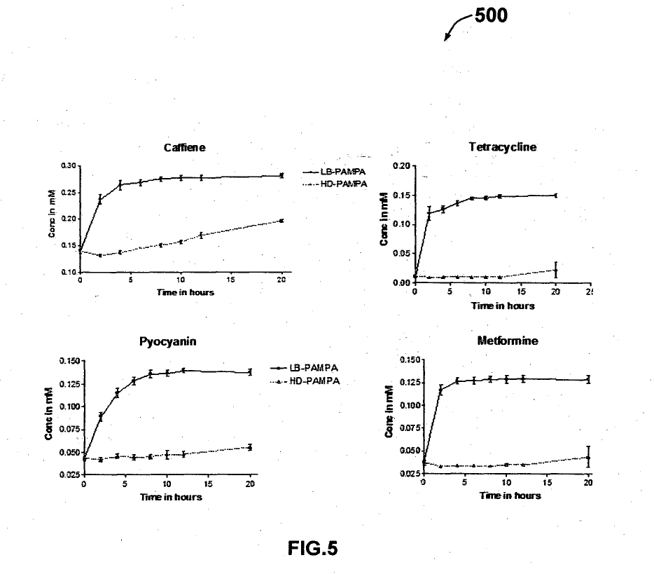

FIG. 5 illustrates a graph 500 illustrating the equilibration time observed in the present system, in accordance with the disclosed embodiments. The graph 500 indicates that the present invention has lesser equilibration time when compared to other PAMPA systems. The system proposed herein also use less amount of lipid compared to the conventional PAMPA systems. The amount of lipid covering the membrane in the present LB model is about 1.2 μg of DPPC for an area of 55.42 mm2, whereas the amount of hexadecane used in HD-PAMPA is 5 μl and in other solvent based models 4 μg of phospholipid dissolved in 5 μl of hexadecane or dodecane is used.

In the present study a comparison of flux through the monolayer based LB-PAMPA and heaxadecane based PAMPA (HD-PAMPA) was carried out. The reduction in the equilibration time observed in the current monolayer based LB-PAMPA is predominantly due to the reduction in the resistance offered by the membrane. Presence of solvent in the pores adds to the resistance of the membrane. Absence of solvent reduces the equilibration time. As shown in Figure it is clear that the HD-PAMPA takes longer time for equilibration whereas LB-PAMPA approaches the equilibrium much faster.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

I/we claim:

1. A method for measuring the permeability of drugs/toxin/NCE's/chemical compounds utilizing phospholipid monolayer coated porous support through Boyden chamber assay, said method comprising:
depositing phospholipid monolayer onto a porous support (e.g.: Cellulose nitrate Membrane) wherein the monolayer is formed by dissolving phospholipid in spreading agent and spread on liquid surface and dried in order to permit the spreading agent to evaporate and the phospholipid is left behind;
compressing the phospholipid molecules using barrier wherein the phospholipid monolayer can be further compressed to attain close packing of phospholipid molecules and allowed to stabilize followed by transfer of monolayer on to porous support; and
attaching the coated porous support to Polystyrene tubes/Poly acrylic tubes and assembling the tubes in from of boyden chamber in order to thereby measure the permeability of the drug/toxin/NCE's/chemical compound using the phospholipid monolayer wherein the permeability is determined by measuring the concentration of drug/toxin/chemical/NCE's in the two compartments.

2. The method of claim 1 wherein the phospholipid monolayer can be be 1,2-dipalmitoyl-sn-glycero-3-phosphocholine (DPPC), pure lipids, fatty acids or mixture or extract of lipids and other fatty acids.

3. The method of claim 1 wherein the spreading agent for phospholipid molecules can be solvent, inorganic solvent like chloroform, hexane, acetone, methanol etc.

4. The method in claim 1 wherein the liquid surface can be water, buffer,mercury

5. The method of claim 1 wherein the phospholipid monolayer can be a transferred onto porous support by vertical lifting or langmuir Schafer deposition or rotary deposition

6. The method of claim 1 wherein the measurement of drug permeability using the phospholipid monolayer can be done with least amount of lipid use and lesser equilibration time.

7. The method of claim 1 wherein the lipid molecules of the proposed invention are arranged in planar monolayer eliminating the solvents by making it similar to Cell Membrane

8. The method of claim 1 wherein the cellulose nitrate membrane support can be used interchanged with other porous supports.

9. The method of claim 1 wherein the permeability of the drug/toxin/chemical compound can be effectively measured using the phospholipid monolayer by Boyden chamber approach.

10. The method of claim 1 wherein coating the monolayer with the membrane is suitable for a wide range of porous substrates including PVDF, Nylon and Polycarbonate membranes.
